(19) United States
(12) Patent Application Publication
(10) Pub. No.: US 2019/0113495 A1
(43) Pub. Date: Apr. 18, 2019

(54) Remote Control and Monitoring Apparatus and Method for Plant Growth

(71) Applicant: Eli Gabay, Las Vegas, NV (US)

(72) Inventor: Eli Gabay, Las Vegas, NV (US)

(21) Appl. No.: 16/164,791

(22) Filed: Oct. 18, 2018

Related U.S. Application Data
(60) Provisional application No. 62/574,033, filed on Oct. 18, 2017.

(51) Int. Cl. G01N 33/00; A01G 27/02; A01G 27/00; G05B 15/02; H04N 7/18; G01N 33/24

(52) U.S. Cl. CPC G01N 33/0098; A01G 27/02; A01G 27/003; H04N 5/2625; G05B 15/02; H04N 7/183; G01N 33/246; A01G 27/008

(57) ABSTRACT

A remote control and monitoring apparatus and method for plant growth has an apparatus for planting and monitoring the growth of various plants, and an accompanying method lays out steps for the operation thereof. A planter body provides a plant receptacle, as well as an irrigation mechanism for watering the plants on a schedule or as otherwise defined, a plurality of sensors for monitoring the environmental conditions of for the plants and alerting the user is any parameters fall out of an acceptable range, and at least one camera to view the plant at any time as well as capture images and timelapses.

The current application claims a priority to the U.S. Provisional Patent application Ser. No. 62/574,033 filed on Oct. 18, 2017.

### FIELD OF THE INVENTION

The present invention relates generally to methods of growing plants and apparatus to aid in the process of plant growth. More specifically, the present invention relates to a new and improved method and apparatus to grow plants.

### BACKGROUND OF THE INVENTION

Growing plants is an activity and hobby practiced by thousands of people around the world. A common practice is to grow different plants on pots or planters which allow users to grow plants outdoors or indoors. Growing plants on pots or planters can be a challenging task as they require the same or more amount or care which would be required by growing plants on gardens. Due to the smaller size of the pot or planter, it is easier to overwater or to not water the plant enough. In addition, without the proper instruments and knowledge, users find it hard to maintain a proper environment for the plants to grow properly and/or to know when to adjust the proper environmental variables, such as the temperature, humidity, or the light intensity. More recently, plant growers have begun to utilize electronic tools to aid in the plant growing process, such as temperature sensors, light sensors, humidity sensors, etc. In addition, some pots or planters have been upgraded to include a system to keep track of the conditions of the growing plant. Some of the new pots or planters have also been fitted with an automatic watering system to help plant growers keep the growing plants watered. However, there is not a pot or plant currently available which integrates a self-watering mechanism as well as an environment control mechanism. Furthermore, there is not a pot or planter which implements a method of automatic environment control and plant care which alerts users when the plant or the device needs attention from the user as well as provides the user with information regarding the plant's growth and conditions. Thus, a pot or planter which provides an automated environment control and plant care as well as implements a method of alerting and providing a user with information regarding the plant's growth and condition is beneficial and necessary.

An objective of the present invention is to provide a method and apparatus for the process of plant growth which provides an automated environment control and plant care system. Another objective of the present invention is to provide a method and apparatus for the process of plant growth which provides a device integrated with an environment control mechanism as well as a plant care mechanism. Another objective of the present invention is to provide a method and apparatus for the process of plant growth which allows users to control and keep track of the plant growth remotely. Another objective of the present invention is to provide a method and apparatus for the process of plant growth which allows users to monitor the plant growth remotely as well as to share the plant growth on a wireless social platform. Another objective of the present invention is to provide a method and apparatus for the process of plant growth which allows an administrator to control a multiple number of devices through a single, unified cloud platform. Additional advantages of the invention will be set forth in part in the description which follows, and in part will be obvious from the description, or may be learned by practice of the invention. Additional advantages of the invention may be realized and attained by means of the instrumentalities and combinations particularly pointed out in the detailed description of the invention section. Further benefits and advantages of the embodiments of the invention will become apparent from consideration of the following detailed description given with reference to the accompanying drawings, which specify and show preferred embodiments of the present invention.

### SUMMARY OF THE INVENTION

The present invention is a method and apparatus for the process of plant growth also known as Mary Grow. The method and apparatus for the process of plant growth is an application and device which aids plant growers through the process of plant growth. The method and apparatus for the process of plant growth helps users in their plant growing operations by monitoring and aiding in the environment in which the apparatus is located. The method and apparatus for the process of plant growth provides users with notifications regarding the conditions of the growing plant. The method and apparatus for the process of plant growth utilizes data obtained from the plurality of sensors and other electrical components on the apparatus to track the conditions and any abnormalities on the growth of the plant. For large scale operations, the method and apparatus for the process of plant growth provides users with a task control and monitoring system which provides management with a platform to assign tasks to employees as well as to keep track of the status of operations. In alternate embodiments of the present invention, the method and apparatus for the process of plant growth can further provide a business platform for users to grow and sell a variety of plants.

### BRIEF DESCRIPTION OF THE DRAWINGS

FIG. 1 is a front perspective view of one embodiment of the present invention.

FIG. 2 is a rear perspective view of one embodiment of the present invention.

FIG. 3 is a rear view of one embodiment of the present invention.

FIG. 4 is a side cross-section view of one embodiment of the present invention.

FIG. 5 is a diagram illustrating the electrical connections of the present invention.

FIG. 6 is a general process flow in one embodiment of the present invention.

FIG. 7 is a general process flow in one embodiment of the present invention.

FIG. 8 is an illustration of a user login screen in one embodiment of the present invention.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

The present invention is a method and apparatus for remote control and monitoring of plant growth and may be alternatively known as Mary Grow. The method and apparatus for the process of plant growth provides an apparatus and automated method of facilitating the growth and care of a variety of plants. The method and apparatus for the process of plant growth allows users to monitor and control the growth of a number of plants remotely as well as keep track of any abnormalities in the system which may pose a danger to the optimal growth of the plants. In the preferred embodiment of the present invention, the method and apparatus for the process of plant growth comprises an apparatus and a method.

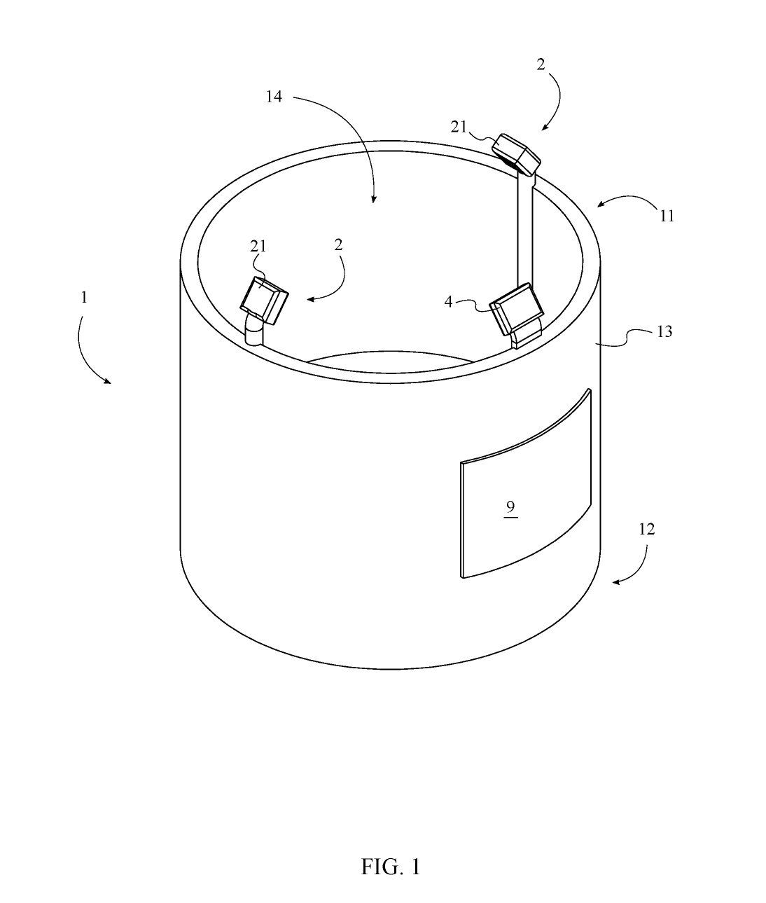

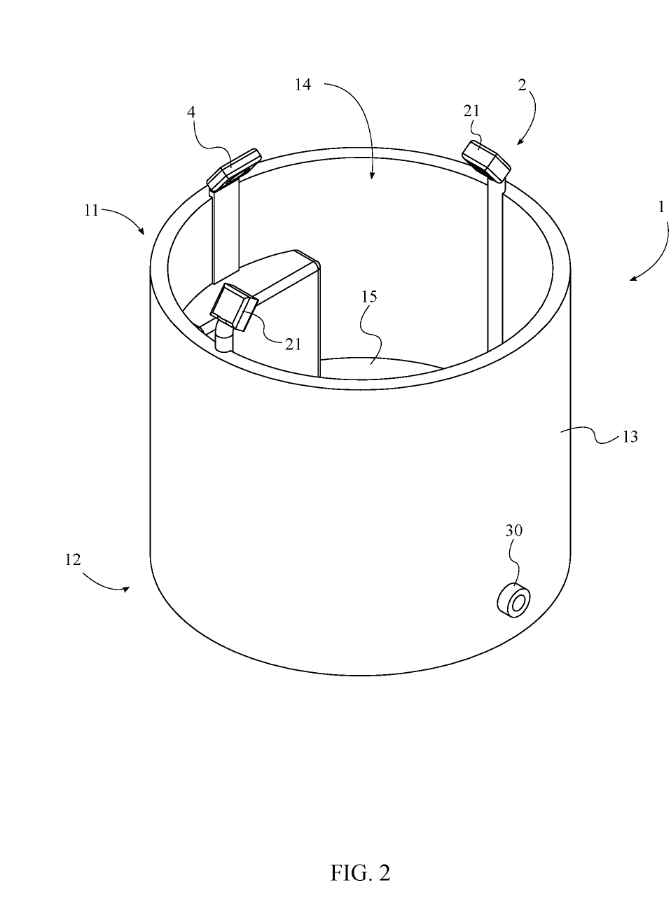

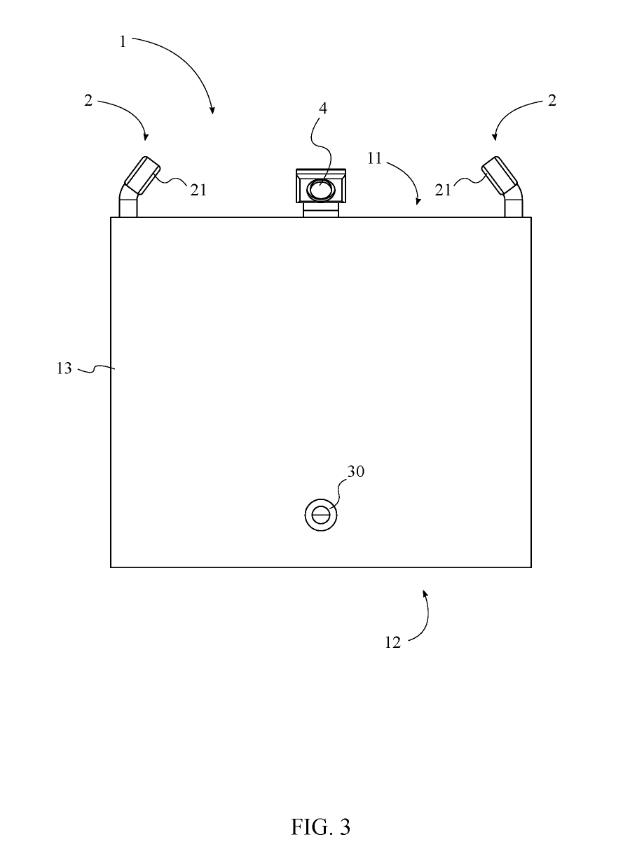

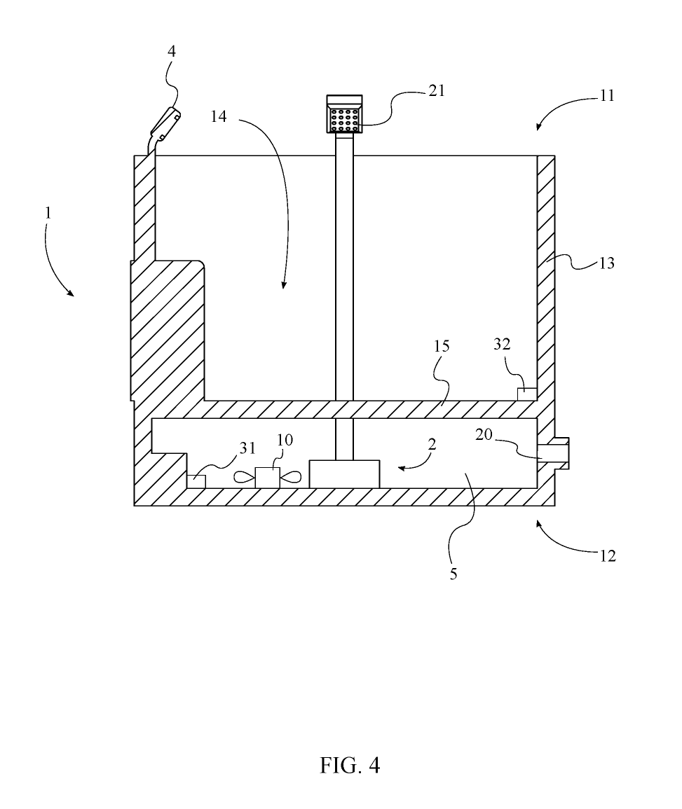

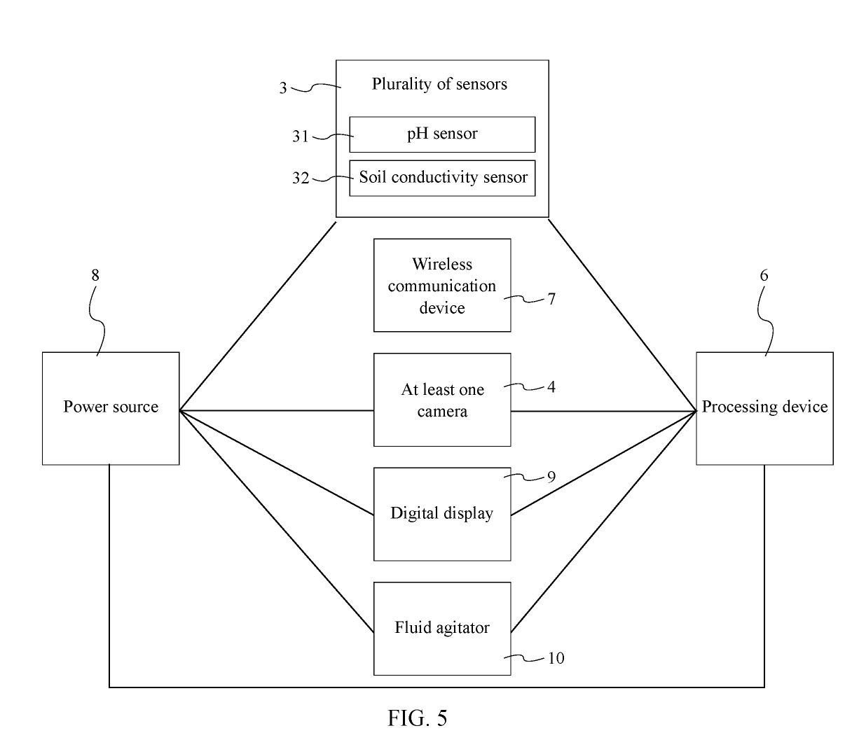

The apparatus of the present invention is a remote control and monitoring apparatus for plant growth. In general, referring to FIGS. 1-5, the apparatus of the present invention is a planter and comprises a planter body 1, an irrigation mechanism 2, a plurality of sensors 3, at least one camera 4, a fluid reservoir 5, a processing device 6, a wireless communication device 7, and a power source 8.

The planter body 1 is the main structure of the apparatus of the present invention and holds any soil and plants placed by the user. In the preferred embodiment of the present invention, the planter body 1 of the apparatus is a thin-walled, generally cylindrical body. In alternate embodiments of the present invention, the body of the apparatus may comprise a different structure and design. In the preferred embodiment of the present invention, the planter body 1 comprises a top end 11, a bottom end 12, a lateral wall 13 and a plant receptacle 14. The plant receptacle 14 is the main space within which the user can place soil, plant seeds, and grow various plants. The plant receptacle 14 is positioned within the lateral wall 13 between the top end 11 and the bottom end 12, and an opening 20 at the top end 11 allows the user to place soil, seed or other items within the plant receptacle 14 and otherwise tend to their plants. The irrigation mechanism 2 is connected to the planter body 1 such that the irrigation mechanism 2 is configured to distribute fluid within the plant receptacle 14. The irrigation mechanism 2 is in fluid communication with the fluid reservoir 5 through a pump, so that any fluid placed by the user within the fluid reservoir 5 is used by the irrigation mechanism 2 to irrigate any plants present within the plant receptacle 14.

The plurality of sensors 3 is positioned about the planter body 1. The composition of the plurality of sensors 3 may vary in different embodiments, and may comprise any sensors relevant to monitoring plants and ensuring their effective growth. The location of any given sensor from the plurality of sensors 3 will depend on the specific type of sensor. The plurality of sensors 3 is positioned around the body of the apparatus and help monitor a plurality of environment variables including, but not limited to, temperature, humidity, and other relevant variables. In the preferred embodiment of the present invention, the plurality of sensors 3 includes, but is not limited to, light sensors, temperature sensors, water level sensors, pH sensors, humidity sensors, etc. In the preferred embodiment, the plurality of sensors 3 comprises at least one pH sensor 31 and at least one soil moisture sensor 32. Preferably, a first pH sensor 31 is positioned within the plant receptacle 14, and a second pH sensor 31 is positioned within the fluid reservoir 5. The at least one soil moisture sensor 32 is positioned within the plant receptacle 14.

The power source 8 is electrically connected to the at least one camera 4, the wireless communication device 7, and the processing device 6. In various embodiments, the power source 8 may vary. In some embodiments, the power source 8 may be a wired plug corresponding to a typical National Electrical Manufacturers Association (NEMA) plug, or any relevant type of connection to a electrical network of a household or other building. In some embodiments, the power source 8 may comprise a battery, which may be removable or non-removable, and which may continue to power the present invention if the wall socket connection fails or the user's main power goes down. The processing device 6 is electronically connected to the at least one camera 4, the plurality of sensors 3, the irrigation mechanism 2, and the wireless communication device 7. The present invention may further comprise one of more lights which are electrically connected to the power source 8 and electronically connected to the processing device 6. The one or more lights may be positioned in any desired configuration around the planter body 1. The at least one camera 4 helps monitor the growing plant visually and allows users to record and take pictures of the growing plant.

In some embodiments, the planter body 1 further comprises a divider 15, wherein the divider 15 is connected within the lateral wall 13 between the top end 11 and the bottom end 12. The fluid reservoir 5 is further positioned within the lateral wall 13 adjacent to the bottom end 12, and the fluid reservoir 5 is separated from the plant receptacle 14 by the divider 15, wherein the plant receptacle 14 is positioned adjacent to the top end 11.

In the preferred embodiment of the present invention, the irrigation mechanism 2 comprises a plurality of sprinklers 21. In other embodiments, the irrigation mechanism 2 may comprise alternate components and arrangements directed toward distributing fluid to any plants within the plant receptacle 14. Preferably, the plurality of sprinklers 21 are distributed about the planter body 1. More particularly, in some embodiments, each of the plurality of sprinklers 21 are positioned on the lateral wall 13 adjacent to the top end 11. Furthermore, each of the plurality of sprinkler is in fluid communication with the fluid reservoir 5 through at least pump which propels fluid from the fluid reservoir 5 through one or more tubes to each of the sprinklers 21.

In the preferred embodiment of the present invention, the control mechanism further comprises a communication system which allows the processing device 6 to wirelessly send and receive data. In alternate embodiments of the present invention, the apparatus of the method and apparatus for the process of plant growth may further comprise additional sensors or electrical components which allows the user to better monitor and control the growth of the plants. The apparatus of the method and apparatus for the process of plant growth may alternatively be solar powered.

In some embodiments, the present invention further comprises a digital display 9 through which the user may interact with the various electrical and electronic components of the planter. One or more display and one or more interface may further be positioned on the outer surfaces of the plurality of walls of the body of the apparatus. The one or more display allows the user to track the data received from the monitoring mechanism as well as to directly control and configure the control mechanism and irrigation mechanism 2 of the apparatus. The digital display 9 is electrically connected to the power source 8, electronically connected to the processing device 6, and externally connected to the planter body 1. The digital display 9 may be used for any number of functions, including, but not limited to, displaying statistics about any current occupants of the plant receptacle 14, controlling parameters for the irrigation mechanism 2, controlling the at least one camera 4, lights, or other components.

The preferred embodiment of the present invention further comprises a fluid agitator 10. The fluid agitator 10 is electrically connected to the power source 8 and electronically connected to the processing device 6. The fluid agitator 10 is positioned within the fluid reservoir 5 in order to ensure that any plant nutrients or other chemicals or compounds introduced to the fluid reservoir 5 by the user are appropriately mixed into the bulk of the water or other fluid contained within the fluid reservoir 5.

The present invention may further comprise an opening 20 traversing through the lateral wall 13 into the fluid reservoir 5 adjacent to the bottom end 12. The opening 20 may be used to introduce new matter, such as additional water, plant nutrients, or other matter, into the fluid reservoir 5, or to drain existing material out of the fluid reservoir 5 as desired. In some embodiments, a cap 30 is removably connected to the opening 20 adjacent to the lateral wall 13 opposite the fluid reservoir 5, exterior to the planter body 1. Alternatively, the opening 20 may comprise a valve or other means to control the flow of fluid or other matter through the opening 20.

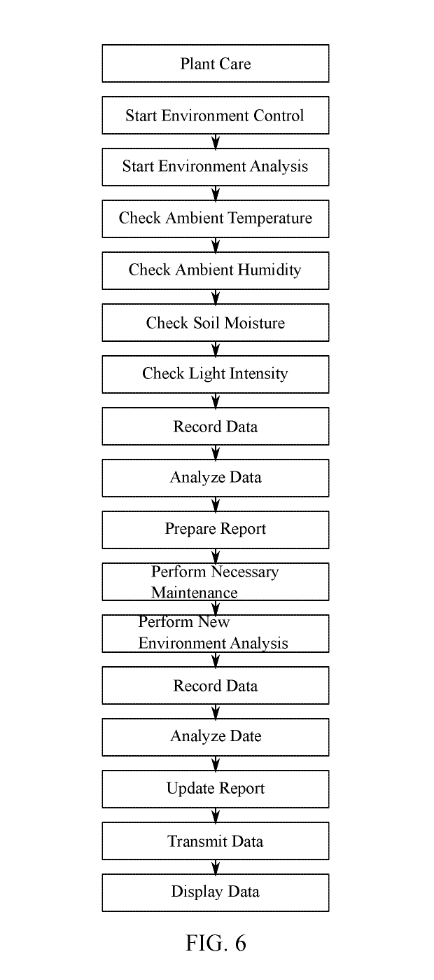

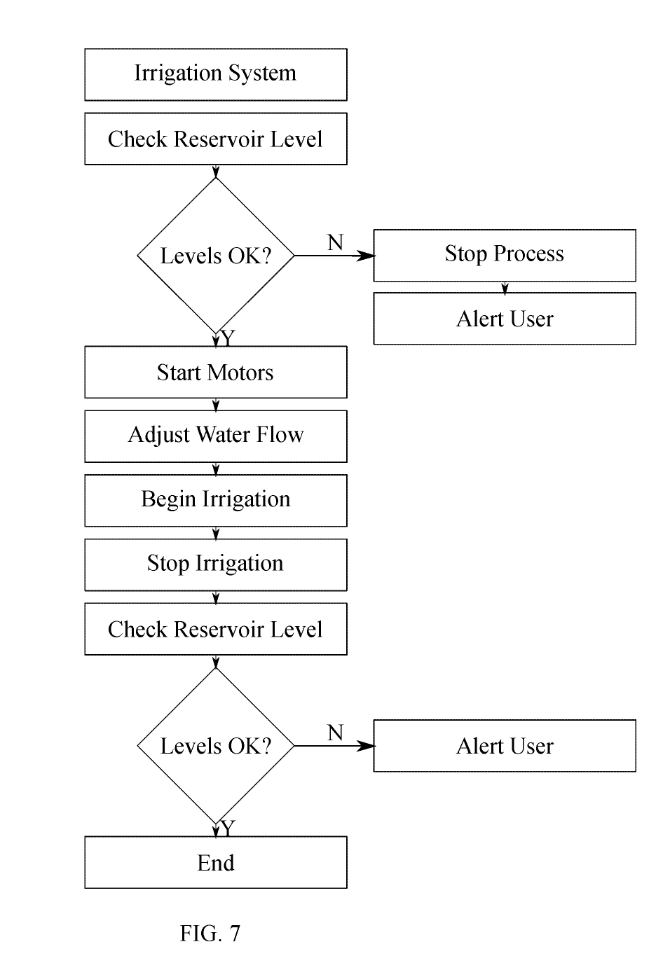

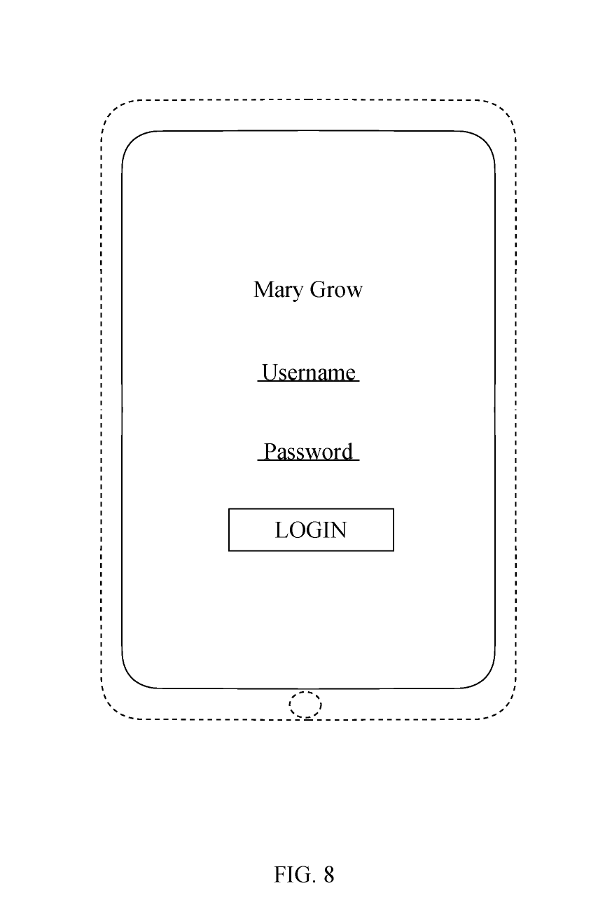

Referring to FIGS. 6-8, the preferred embodiment of the present invention further comprises a method of utilizing and controlling the apparatus to optimize the growth of the plants. In the preferred embodiment of the present invention, the application of the method may be a software application, mobile application, cloud application, add-on, plug-in, website, etc. The application of the method may be installed on a device which may be wireless or hardwired and can connect to the Internet wirelessly or hard-wired. The application of the method allows the user to access a one or more database. The one or more database is preferably hosted by Microsoft Azure. The one or more database holds users' information, apparatus name and information, and other user specific data. Another separate one or more database may hold apparatus general data and sensors data. In the preferred embodiment of the present invention, only authorized users will have access to the one or more database through the application. The preferred embodiment of the method of the present invention, further allows users to retrieve pictures or recordings of the growing plants and share them on a social platform. The application allows users to customize and modify the pictures and recordings of the growing plants in order to be shared on a social platform. The control system of the method and apparatus for the process of plant growth allows users to automate and remotely control the growth of the plants.

In the method of operating the remote control and monitoring apparatus of the present invention, the processing device 6 is provided, in addition to a user device, the plurality of sensors 3, the at least one camera 4, and the irrigation mechanism 2. The method of the present invention may further provide a mobile and/or desktop application for interaction with and control of the planter apparatus.

A plurality of signals is received from the plurality of sensors 3 with the processing device 6. The plurality of signals may be automatically sent from the sensors, or the processing device 6 may query the sensors in various embodiments. The plurality of signals is analyzed with the processing device 6 in order to determine if each of the plurality of signals is within a specified acceptable range for each of the plurality of signals. An alert is sent to the user device, if the plurality of signals is not within the specified acceptable range. Furthermore, the method of the present invention provides various means of controlling the apparatus of the present invention. For example, watering of any plants present in the plant receptacle 14 may be automated through the method of the present invention. To this end, an activation signal may be periodically sent to the irrigation mechanism 2 through the processing device 6 according to an irrigation schedule. The irrigation schedule may be predefined according to a plant type designation provided by the user or the irrigation schedule may be user defined. Furthermore, a fluid agitator 10 may be provided, whereby the fluid agitator 10 may be activated to mix fluid within the fluid reservoir 5 by sending an activation signal to the fluid agitator 10 through the processing device 6.

Preferably, the present invention allows the user to remotely monitor their plants through the at least one camera 4 of the present through invention through a mobile application or other user interface. Thus, a live video stream is received by the processing device 6 from the at least one camera 4, and the live video stream may be sent to the user device upon request, and the live video stream is then displayed on the user device.

As mentioned, the present invention monitors the various sensors employed by the present invention in order to determine if conditions are appropriate for optimal plant growth. To this end, in the preferred embodiment of the present invention, the plurality of sensors 3 comprises at least one pH sensor 31. At least one pH signal is received as one of the plurality of signals, and an alert is sent to the user device if the at least one pH signal is not within a specified acceptable pH range. Similarly, the plurality of sensors 3 comprises at least one soil conductivity sensor in the preferred embodiment. At least one soil conductivity signal is received as one of the plurality of signals, and an alert is sent to the user device if the at least one soil conductivity signal is not within a specified acceptable soil conductivity range.

The mobile application of the present invention may provide the user with information related to the care of various plants. Thus, the user may enter a plant specification selection through the user device, which corresponds to a plant placed within the plant receptacle 14 by the user. A plurality of plant information corresponding with the plant specification selection is retrieved from a plant database, and the plurality of plant information is then displayed on the user device. The plant information may include, but is not limited to, optimal watering schedule, required or optional nutrient additives, and more.

Furthermore, the user is provided with the ability to produce a timelapse of the growth of their plant through the method of the present invention. A plurality of images is captured through the at least one camera 4 according to a timelapse interval. The plurality of images is combined into a timelapse video, and the timelapse video may be displayed on the user device at the user's request. Furthemore, the timelapse video and any other captures images or videos may be saved and shared to an online user profile.

In the preferred embodiment of the present invention, the user can receive notifications from the apparatus on the application installed on a wireless device. In alternate embodiments of the present invention, the method of the method and apparatus for the process of plant growth further comprises additional systems for the automation and remote control of the apparatus to optimize the growth of the plant and to prevent or fix any malfunctions/abnormalities. The method and apparatus for the process of plant growth may further comprise an integrated social platform for users to share content related to the growth of plants and the experience of growing plants.

In the preferred embodiment of the present invention, the method and apparatus for the process of plant growth further comprises a business platform and management platform. The business platform of the method and apparatus for the process of plant growth can be integrated on the application to allow users to sell the plants. The business platform may offer a plurality of services including, but not limited to, selling, digital dashboards, performance management, document management, etc. The management platform may offer a plurality of management services such as, but not limited to, task management, scheduling, etc. The method and apparatus for the process of plant growth allows individuals and company growing operations by monitoring and aiding in the environment on which the plants are growing. The application will send notifications to users regarding malfunctions or abnormalities on the growth of the plant which may harm the optimal growth of plant. The application can be used to assign task as well as send messages to employees as well as to customize and set alerts for their preference with regards to the entire plant system. The application may comprise a log-in page where the user can securely log into the application utilizing secure credentials. Preferably, the application further comprises a homepage where users are provided a dashboard which may include, but is not limited to, important notifications, plant status, etc. The application may further comprise a swipe or slide menu which can be accessed by swiping or sliding the interface of the device to a side. The application may further comprise a garden page where the user can check the plurality of plants conditions. The garden page can provide various views including, but not limited to, data list views, visual graphic views, graphical views, etc. The application may further comprise a settings page where the user can perform various tasks including, but not limited to, manage users' accounts, profile settings, learn about the present invention, get help about the present invention, etc. The users will have the option to sell plants or appoint the ability to sell plants using the point of sale system inside the application. The method and apparatus for the process of plant growth further provides a system for growth comparison between prior harvests and current harvests. The users may utilize the method and apparatus for the process of plant growth to determine the best conditions to grow their plants utilizing the data history. Utilizing the data history, the users can determine and set up the optimal environment for their plants and harvests. In alternate embodiments of the present invention, the method and apparatus for the process of plant growth further provides a unique algorithm which utilizes data history from previous harvests and current plant conditions to set the most optimal growing conditions for future harvests.

Although the invention has been explained in relation to its preferred embodiment, it is to be understood that many other possible modifications and variations can be made without departing from the spirit and scope of the invention as hereinafter claimed.

What is claimed is:

1. A remote control and monitoring apparatus for plant growth comprises:
a planter body;
an irrigation mechanism;
a plurality of sensors;
at least one camera;
a fluid reservoir;
a processing device;
a wireless communication device;
a power source;
the planter body comprises a top end, a bottom end, a lateral wall and a plant receptacle;
the plant receptacle being positioned within the lateral wall between the top end and the bottom end;
the irrigation mechanism being connected to the planter body, wherein the irrigation mechanism is configured to distribute fluid within the plant receptacle;
the irrigation mechanism being in fluid communication with the fluid reservoir;
the plurality of sensors being positioned about the planter body;
the power source being electrically connected to the at least one camera, the wireless communication device, and the processing device; and
the processing device being electronically connected to the at least one camera, the plurality of sensors, the irrigation mechanism, and the wireless communication device.

2. The remote control and monitoring apparatus for plant growth as claimed in claim 1 comprises:
the planter body further comprises a divider;
the divider being connected within the lateral wall between the top end and the bottom end;
the fluid reservoir being positioned within the lateral wall adjacent to the bottom end; and
the fluid reservoir being separated from the plant receptacle by the divider, wherein the plant receptacle is positioned adjacent to the top end.

3. The remote control and monitoring apparatus for plant growth as claimed in claim 1 comprises:
the plurality of sensors comprises at least one pH sensor.

4. The remote control and monitoring apparatus for plant growth as claimed in claim 1 comprises:
the plurality of sensors comprises at least one soil moisture sensor.

5. The remote control and monitoring apparatus for plant growth as claimed in claim 1 comprises:
the irrigation mechanism comprises a plurality of sprinklers;
the plurality of sprinklers being distributed about the planter body; and
each of the plurality of sprinklers being in fluid communication with the fluid reservoir.

6. The remote control and monitoring apparatus for plant growth as claimed in claim 5 comprises:
each of the plurality of sprinklers being positioned on the lateral wall adjacent to the top end.

7. The remote control and monitoring apparatus for plant growth as claimed in claim 1 comprises:
a digital display;
the digital display being electrically connected to the power source;
the digital display being electronically connected to the processing device; and
the digital display being externally connected to the planter body.

8. The remote control and monitoring apparatus for plant growth as claimed in claim 1 comprises:
a fluid agitator;
the fluid agitator being electrically connected to the power source;
the fluid agitator being electronically connected to the processing device; and
the fluid agitator being positioned within the fluid reservoir.

9. The remote control and monitoring apparatus for plant growth as claimed in claim 1 comprises:
an opening; and
the opening traversing through the lateral wall into the fluid reservoir.

10. The remote control and monitoring apparatus for plant growth as claimed in claim 9 comprises:
a cap; and
the cap being removably connected to the opening adjacent to the lateral wall opposite the fluid reservoir.

11. A method of operating a remote control and monitoring apparatus for plant growth:
providing a processing device, a user device, a plurality of sensors, at least one camera, and an irrigation mechanism;
receiving a plurality of signals from the plurality of sensors with the processing device;
analyzing the plurality of signals with the processing device in order to determine if each of the plurality of signals is within a specified acceptable range;
sending an alert to the user device, if one of the plurality of signals is not within the specified acceptable range;
periodically sending an activation signal to the irrigation mechanism through the processing device according to an irrigation schedule;

12. The method of operating a remote control and monitoring apparatus for plant growth as claimed in claim 11 comprises the steps of:
receiving a live video stream from the camera;
sending the live video stream to the user device;
displaying the live video stream on the user device;

13. The method of operating a remote control and monitoring apparatus for plant growth as claimed in claim 11 comprises the steps of:
wherein the plurality of sensors comprises at least one pH sensor;
receiving at least one pH signal from the at least one pH sensor as one of the plurality of signals;
sending an alert to the user device, if the at least one pH signal is not within a specified acceptable pH range;

14. The method of operating a remote control and monitoring apparatus for plant growth as claimed in claim 11 comprises the steps of:
wherein the plurality of sensors comprises at least one soil conductivity sensor;
receiving at least one soil conductivity signal from the at least one pH sensor as one of the plurality of signals;
sending an alert to the user device, if the at least one soil conductivity signal is not within a specified acceptable soil conductivity range;

15. The method of operating a remote control and monitoring apparatus for plant growth as claimed in claim 11 comprises the steps of:
providing a fluid agitator;
sending an activation signal to the fluid agitator through the processing device;

16. The method of operating a remote control and monitoring apparatus for plant growth as claimed in claim 11 comprises the steps of:
receiving a plant specification selection through the user device;
retrieving a plurality of plant information corresponding with the plant specification selection from a plant database;
displaying the plurality of plant information on the user device;

17. The method of operating a remote control and monitoring apparatus for plant growth as claimed in claim 11 comprises the steps of:
periodically capturing a plurality of images through the at least one camera according to a timelapse interval;
combining the plurality of images into a timelapse video;
displaying the timelapse video on the user device;
